SEQUENCE LISTING (1 sequence)

SEQ ID NO: 1 - protein, 41 aa, Drosophila melanogaster
NLQQQRQHGKNGNQDYQDQSNEQRKNQRTSSEEDYSEEVKN

(19) United States
(12) Patent Application Publication
(10) Pub. No.: US 2025/0327025 A1
(43) Pub. Date: Oct. 23, 2025

(54) Remote Methods and Elements for Genetic Modification of Insects

(71) Applicant: BYBUG INC., COQUIMBO (CL)

(72) Inventors: DANIEL TRONCOSO ESPINOSA, COQUIMBO (CL); JOSÉ IGNACIO DEL SOLAR BOU, COQUIMBO (CL); GERARDO BLUSKE MOSCOSO, COQUIMBO (CL); ROCÍO DEL CARMEN ESPINOSA TORRES, COQUIMBO (CL); RICARDO FELIPE GUTIERREZ GARCIA, COQUIMBO (CL); VALENTINA MUÑOZ ZAPATA, COQUIMBO (CL); FRANCISCO JARA MONTECINOS, COQUIMBO (CL); MACARENA URIARTE OSSANDÓN, COQUIMBO (CL); FRANCISCO JAVIER ALTIMIRAS GONZALEZ, COQUIMBO (CL); MATÍAS GERMÁN BUSCHMANN ESPINOZA, COQUIMBO (CL); MARTHA ADRIANA HAASE MALIG, COQUIMBO (CL); DANIELA LORENA LEIVA CARVAJAL, COQUIMBO (CL); CLARA ANDREA SOLARI, VALPARAISO (CL)

(73) Assignee: BYBUG INC., COQUIMBO (CL)

(21) Appl. No.: 18/764,201

(22) Filed: Jul. 4, 2024

(30) Foreign Application Priority Data
Jul. 4, 2023 (CL) 202301975

(51) Int. Cl. C12N 5/07; C07K 14/435; C12N 9/22; C12N 15/85

(52) U.S. Cl. CPC C12N 5/0601; C07K 14/43581; C12N 9/226; C12N 15/85; C07K 2319/80; C12N 2510/00; C12N 2830/50

(57) ABSTRACT

The present invention relates to the technical field of genetic transformation of insect eggs. Specifically, the present invention refers to an efficient genetic editing system to obtain recombinant or genetically modified insect eggs, by incorporating genetic material directly into oocytes of female insects, which will then generate a large number of eggs with the incorporated or recombinant genetic material.

This application claims priority to Chilean Provisional Application 20/230,1975, filed on Jul. 4, 2023, the entire contents of which are incorporated by reference in its entirety.

### INCORPORATION BY REFERENCE STATEMENT

This application incorporates by reference the sequence listing having the file name 4830BI02UTL1US_2025 Jan. 31.xml created Jan. 31, 2025 with a file size of 1,955 bites and having been filed in the United States Patent and Trademark Office via EFS Web on Feb. 7, 2025.

### TECHNICAL FIELD OF THE INVENTION

The present invention relates to the technical field of genetic transformation of insect eggs.

More specifically, the present invention relates to an efficient gene editing system to obtain recombinant or genetically modified insect eggs, by incorporating genetic material directly into oocytes of female insects, which will then generate a large number of eggs with the incorporated or recombinant genetic material.

### BACKGROUND OF THE INVENTION

The genetic transformation of insect eggs is traditionally carried out through microinjection techniques that allow the introduction of genetic material, and specifically known genetic transformation systems such as CRISPR Cas9, among others, into said eggs. These techniques are tedious since they involve injecting a large number of insect eggs, each one separately, and they also require the participation of highly qualified operators to carry them out, which translates into high costs.

In view of the drawbacks associated with insect genetic transformation techniques using egg microinjection, Chaverra-Rodriguez and collaborators (Chaverra-Rodriguez et al., 2018. Targeted delivery of CRISPR-Cas9 ribonucleoprotein into arthropod ovaries for heritable germline gene editing. Nat Commun. 9 (1): 3008) developed a system called “ReMOT Control” (“Receptor-Mediated Ovarian Load Transduction”) that allows the selective administration of the CRISPR-Cas9 system in arthropod ovaries for the hereditary gene editing of the germ line.

The “ReMOT Control” system is based on the binding of a peptide called P2C, consisting of the minimal functional sequence of the Drosophila Yolk 1 protein, capable of binding to specific receptors on the surface of insect oocytes and the Cas protein of the CRISPR-Cas9 system. The P2C peptide allows the CRISPR Cas9 system to be directed to the surface of female insect oocytes, causing its binding to surface receptors and its subsequent introduction into said oocytes.

The technique allows any protein that has the minimal functional sequence P2C to be directed into an oocyte.

Genetic modification of the germline of female insects allows multiple recombinant eggs to be generated in a single step, unlike microinjection techniques. This represents economic advantages, since there is no need for large numbers of microinjections carried out by expert operators. It also results in operational advantages, since fewer attempts are necessary and therefore efficiency is greater.

However, the transformation efficiency of methods based on the combination of the P2C peptide with other proteins is inversely proportional to the size of said proteins. For this reason, the ReMOT Control system developed by Chaverra-Rodriguez and collaborators, which involves the binding of the P2C peptide to Cas proteins of the large CRISPR-Cas9 system, tends to show a low transformation efficiency. It is with this and other deficiencies in mind that the present invention was developed.

### SUMMARY OF THE INVENTION

The present invention comprises a novel system with improved efficiency for transforming female insect oocytes and generating recombinant eggs.

### BRIEF DESCRIPTION OF THE DRAWINGS

The accompanying drawings are included to provide a greater understanding of the invention and are part of this description and show one or more of the alternate embodiments.

FIG. 1 shows a scheme illustrating the modification of the P2C peptide to improve its interaction with the vitellogenin receptor of a female insect.

FIG. 2 shows a scheme illustrating the replacement of the Cas9 protein of the ReMOT Control system by the HypBase transposase system (piggyBac or hyperBac system).

FIG. 3 shows schemes for 3 embodiments of the present invention that include the use of transposase as a gene editing system.

FIG. 4 shows the embodiment that uses the Cas9 (dead)-pBas P2C fusion protein in combination with the transposase gene editing system.

FIG. 5 shows the “pET28a P2C-HypBase” expression plasmid.

FIG. 6 shows the “pET28a P2C-cas9-HypBase” expression plasmid.

FIG. 7 shows the “pET28a P2C-cas9-HypBase” expression plasmid, which comprises the gene construct that allows the expression of the Cas9 (dead)-pBas-P2C fusion protein.

FIG. 8 shows the “VB230228-1361btg” plasmid as an example of a plasmid with ITR sequences and recombinant protein genes.

FIG. 9 shows a depiction of a tuberculin or insulin syringe.

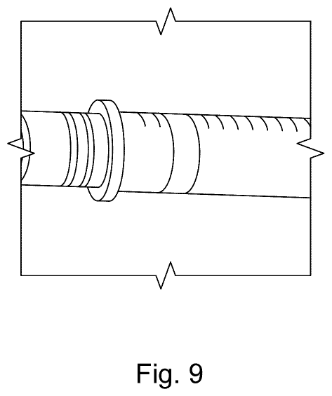

FIG. 10 shows a depiction of the comparison between the bevel and tip diameters of the various needles with A representing a Tuberculin or insulin syringe, B representing a Hamilton needle, and C representing a capillary generated in the laboratory shown with a metric scale.

FIG. 11 depicts the anatomical comparison of a black soldier fly wherein the left view represents the fly with the abdomen facing upwards and the right view represents the fly with the abdomen facing downwards with both figures showing key anatomical features being labeled, including antennae, eyes, wings, scutum, scutellum, anterior abdominal translucent window, posterior abdominal translucent window, and genitalia.

FIG. 12 depicts in the left view an adult female (F) and male (M) black soldier flies (Hermetia illucens); wherein the right view depicts magnified genitals shown with scale bars that are 1 mm in length. Adapted from Oonincx, D., Volk, N., Diehl, J., Van Loon, J., & Belušič, G. (2016). Journal of Insect Physiology, 95, 133-139.

FIG. 13 depicts an adult black soldier fly (Hermetia illucens) injected with fuchsia dye shown with the stained posterior window indicating the successful delivery of the dye into the fly's abdomen.

FIG. 14 depicts an adult black soldier fly (Hermetia illucens) showing dye distribution, wherein the dyed anterior window and dyed legs indicate successful injection and dissemination of the fuchsia dye throughout the body.

FIG. 15 depicts Kaplan-Meier Survival Curves for Injected vs Control Hermetia illucens showing a slight increase in the mortality rate around days 5 and 6 for the injected group compared to the control group.

FIG. 16 depicts the daily mortality rate of female flies with the green bars representing the injected group, and the blue bars representing the control group, with line graphs overlaying the bars to show the cumulative trend of the mortality rate over time.

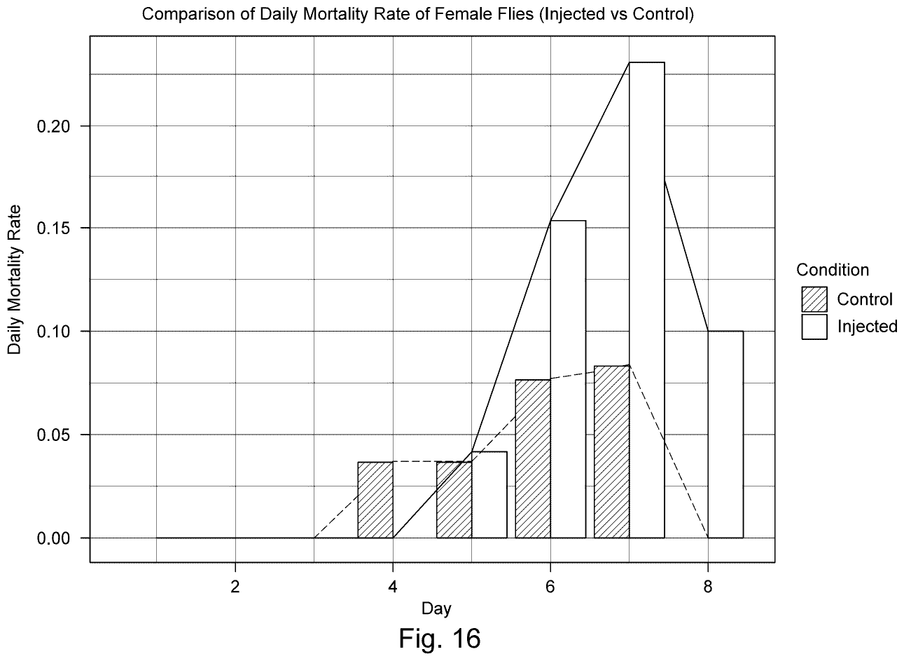

FIG. 17 depicts a bar graph showing the relative death amounts of Hermetia illucens for based upon group and sex with the blue representing males and green representing females.

FIG. 18 depicts differences in antenna coloration at day zero (left) and two days after injection (right).

FIG. 19 depicts hemolymph stained several days after injection.

FIG. 20A-C depict A. one hour post-injection with blue dye. B. fly bladder and genitalia with fuchsia dye from the previous day. C. digestive system and genitalia of a fly injected three days prior.

### DETAILED DESCRIPTION OF THE INVENTION

The present invention provides for a system for transforming female insect oocytes and generating recombinant eggs.

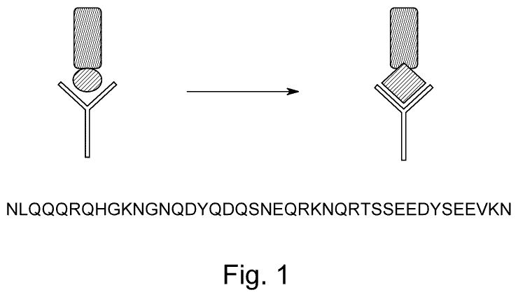

In one embodiment of the present invention, the known P2C peptide, made up of 41 amino acids, is modified by introducing one or more amino acid insertions, deletions and/or changes, among other changes, to optimize its interaction with the vitellogenin receptor of a female insect, and it fuses to, that is, it physically binds, the Cas9 protein to generate a fusion protein comprising the optimized P2C peptide and the Cas9 protein. This embodiment is outlined in FIG. 1. The fusion protein is injected into adult female insects for transformation. In an alternate embodiment of the invention, the changes described in the previous paragraph are introduced to the P2C peptide to optimize its interaction with the vitellogenin receptor of a female black soldier fly.

In another embodiment of the present invention, a fusion protein is generated, called pBas-P2C, which comprises the P2C peptide comprising the minimal functional sequence of the Drosophila Yolk 1 protein, capable of interacting with specific receptors in the surface of insect oocytes, bound to a transposase. Said fusion protein is combined with a genetic vector that comprises inverted terminal repeat (or ITR) sequences and both elements are injected into adult female insects for transformation.

In an alternate embodiment of the present invention, the transposase is a HypBase transposase, which is part of the piggyBac or hyperBac gene editing systems.

In yet another alternate embodiment of the present invention, the genetic vector comprising inverted terminal repeat (or ITR) sequences further comprises a gene that encodes some recombinant protein of interest located between the ITR sequences. The presence of the gene that encodes some recombinant protein of interest in the genetic vector, between the ITR sequences, allows its insertion into the genome of insect oocytes.

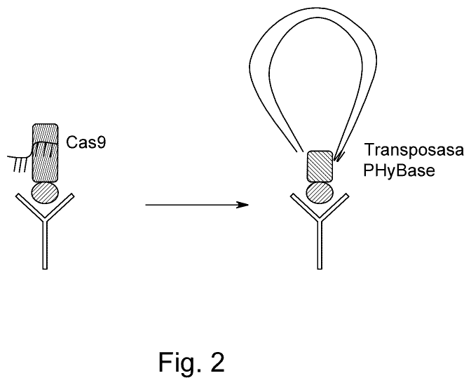

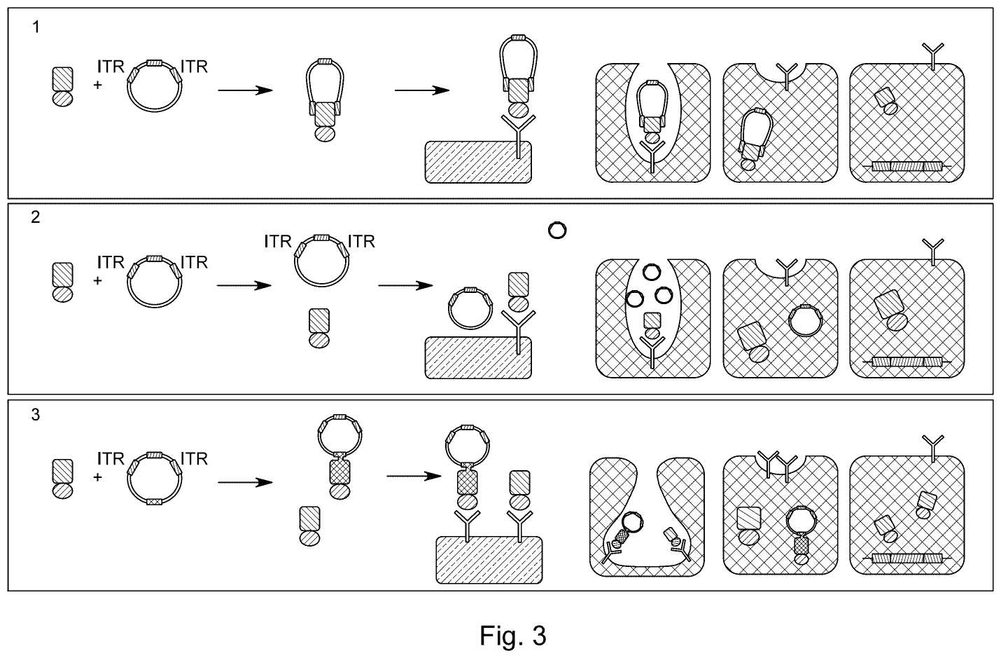

FIG. 2 and points 1 and 2 of FIG. 3 outline embodiments of the present invention that use transposase as a gene editing system. An alternative is shown in point 1 of FIG. 3 in which the genetic vector comprising ITR sequences and a recombinant protein gene forms a complex with the pBas P2C fusion protein, which is actively incorporated completely into the female insect oocyte via endocytosis induced by the interaction between the P2C peptide and the vitellogenin receptors. An alternative is shown in point 2 of FIG. 3 in which the genetic vector comprising ITR sequences and a recombinant protein gene does not form a complex with the pBas-P2C fusion protein, and each element enters the oocyte through different pathways, a passive pathway in the case of the genetic vector, and an active pathway via endocytosis induced by the interaction between the P2C peptide and the vitellogenin receptors, in the case of the pBas-P2C fusion protein.

In yet another embodiment of the present invention, the genetic vector that comprises inverted terminal repeat (or ITR) sequences, and that may comprise a gene that encodes some recombinant protein of interest located between the ITR sequences, further comprises a protein binding sequence. In this embodiment, the system also includes a protein capable of binding to the protein binding sequence included in the genetic vector, which, in turn, is fused to the P2C peptide, that is, a fusion protein capable of binding to the protein binding sequence of the genetic vector and containing the P2C peptide capable of interacting with the vitellogenin receptor of female insect oocytes (pX-P2C fusion protein). This allows a complex to be generated between the genetic vector and the pX-P2C fusion protein, and for both the pBas-P2C fusion protein and the complex between the genetic vector and the pX-P2C fusion protein to enter the oocyte via endocytosis induced by the interaction between the P2C peptide and the vitellogenin receptors. This embodiment is outlined in point 3 of FIG. 3.

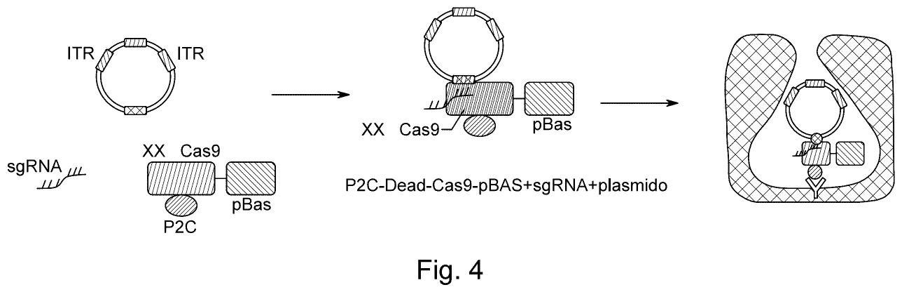

In yet another embodiment of the present invention, a fusion protein is generated that comprises the P2C peptide, a Cas protein and the transposase, called Cas9-pBas P2C. In this embodiment, the genetic vector that comprises inverted terminal repeat (or ITR) sequences is used, in conjunction with the Cas9-pBas-P2C fusion protein and other elements of the CRISPR-Cas9 system such as the guide RNA (gRNA or sgRNA), which may comprise a gene encoding some recombinant protein of interest located between the ITR sequences, and which may further comprise a protein binding sequence. In one embodiment of the present invention, the genetic vector may form a complex with the Cas9-pBas-P2C fusion protein, which is incorporated into the oocyte by active means via endocytosis induced by the interaction between the P2C peptide and the vitellogenin receptors. The complex between the genetic vector and the fusion protein may be generated, among other ways, by the interaction of the gRNA or sgRNA (bound to Cas9) and a protein binding sequence in said vector complementary to the gRNA or sgRNA. This embodiment allows the recombinant protein gene to be inserted into specific places in the genome of the female insect oocyte and is outlined in FIG. 4. In an alternate embodiment, the Cas9 protein may be an inactivated mutated version, called “dead mutant” or “Cas9 (dead)”. The specificity is given by the Cas9 protein, which, in its inactivated mutant version, does not cut the genomic DNA, however, it directs the complex to a specific part of it.

In an alternate embodiment of the present invention, the genetic vector comprising inverted terminal repeat (or ITR) sequences, and a gene that encodes some recombinant protein of interest located between the ITR sequences, further comprises DNA sequences that allow to induce or promote the expression of the gene that encodes some recombinant protein, that is, an expression system.

In an alternate embodiment of the present invention, the genetic vector is constructed based on the commercial vector pET28a.

In an alternate embodiment of the present invention, the genetic material, such as genetic vectors and/or proteins that are part of the systems of said invention, is injected directly into the vitellogenic zone of adult females for transformation.

In one embodiment of the present invention, the injection of genetic material, such as genetic vectors and/or proteins and/or fusion proteins and/or complexes formed by proteins and genetic vectors, is carried out in the vitellogenic zone of adult females for transformation.

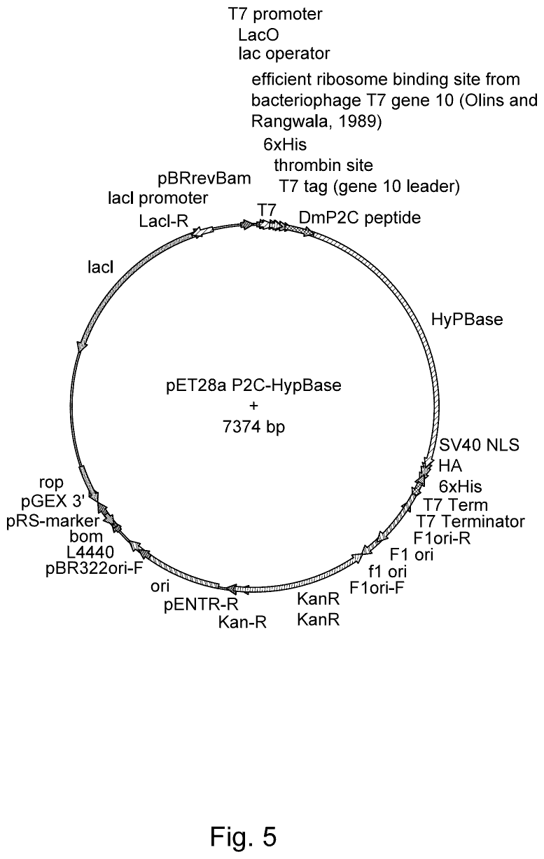

In one embodiment of the present invention, the pBas-P2C fusion protein is obtained by cloning and expressing the “pET28a P2C-HypBase” plasmid constructed based on the commercial plasmid pET28a, and described in FIG. 5, and subsequent purification of the protein.

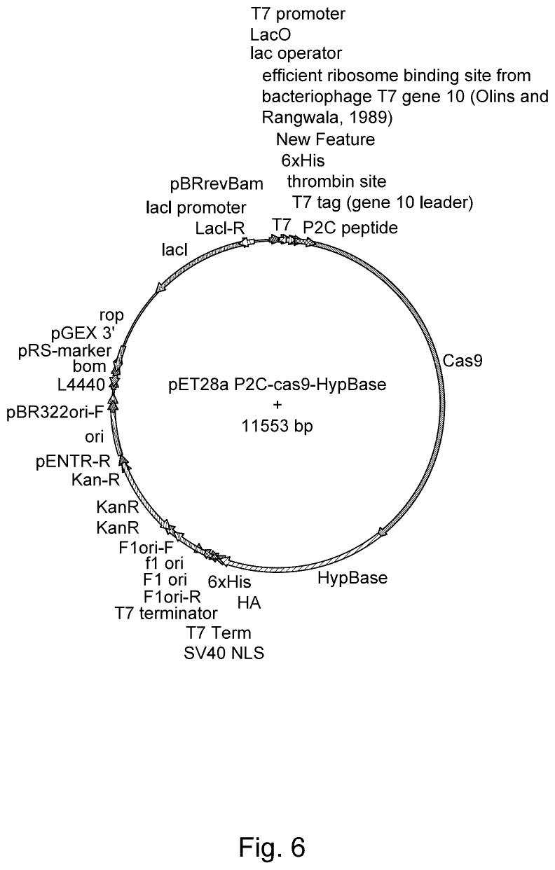

In one embodiment of the present invention, the Cas9-pBas-P2C fusion protein is obtained by cloning and expressing the “pET28a P2C-cas9-HypBase” plasmid constructed based on the commercial plasmid pET28a, and described in FIG. 6, and subsequent purification of the protein.

DmP2C peptide: refers to a peptide sequence of yolk protein 1 that is recognized by the receptor in oocytes. Cas9: refers to an RNA-guided DNA endonuclease enzyme. HyPBase: refers to an improved version of the piggybac transposase.

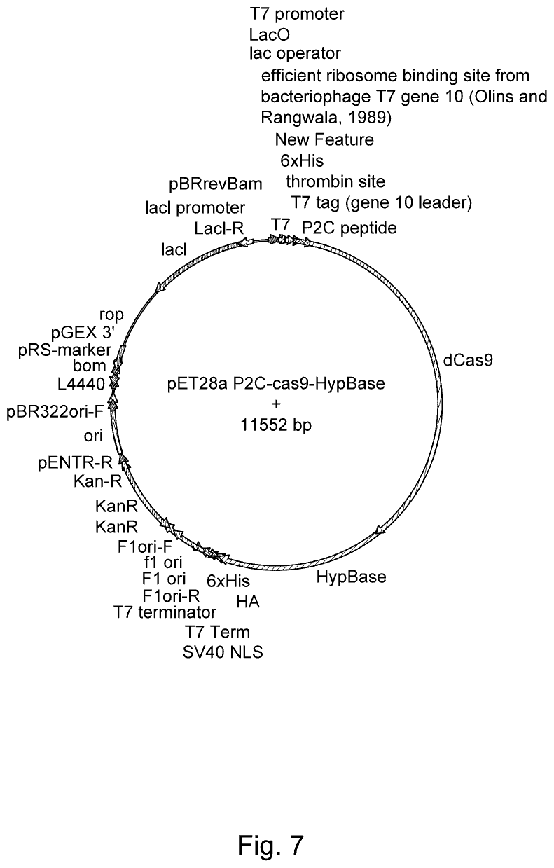

In one embodiment of the present invention, Cas9 (dead)-pBas P2C fusion protein is obtained by cloning and expressing the “pET28a P2C-cas9 HypBase” plasmid constructed based on the commercial plasmid pET28a, and described in FIG. 7, and subsequent purification of the protein.

DmP2C peptide: refers to peptide sequence of yolk protein 1 that is recognized by the receptor in oocytes. dCas9: refers to an RNA-guided DNA endonuclease enzyme with mutations in its active cutting sites (dead activity). HyPBase: refers to an improved version of piggybac transposase.

In one embodiment of the present invention, among other possible embodiments, the genetic vector comprising inverted terminal repeat (or ITR) sequences and a gene that encodes some recombinant protein of interest located between the ITR sequences, may comprise one or more of a 5′ ITR sequence, a promoter sequence “{such as pHi.U6.1}”, a translation initiation sequence “Kozak” that facilitates the initiation of translation from a downstream start codon, sequences that encode self-cleavage peptide sequences such as T2A (dme) or 2A, or P2A of Thosea asigna virus, among others, that facilitate the processing of polycistronic RNA, and a polyadenylation signal sequence, which allows termination of transcription and messenger RNA polyadenylation, an antibiotic resistance marker gene, and a replication origin sequence.

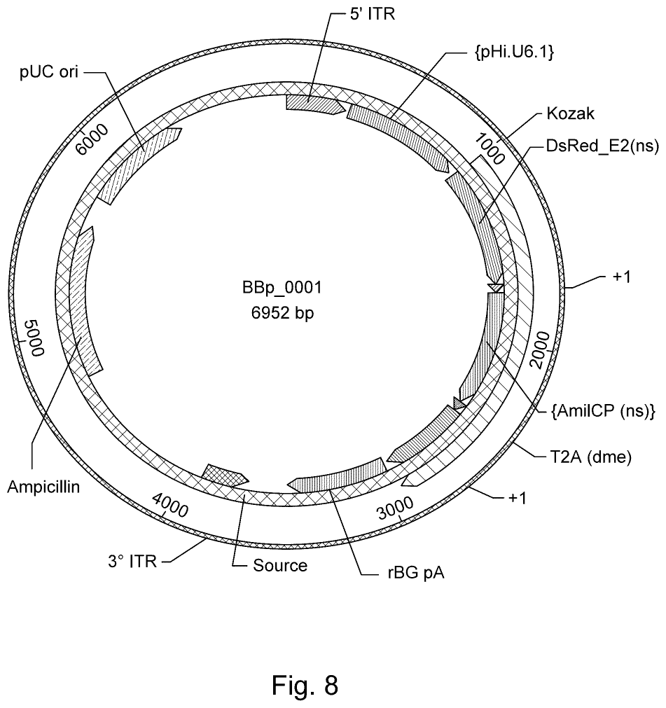

In an alternate embodiment of the present invention, some of these elements may be organized in the plasmid structure called “VB230228-1361btg” as shown in FIG. 8, which has as examples of recombinant genes the sequences DsRed_E2 (ns), {AmilCP (ns)}, {IL22 (optDme) (SCSC)} and where the “5′ ITR” and the “3′ ITR” are ITR sequences of the piggyBac system, “{pHi.U6.1}” is a promoter sequence, “Kozak” is a Kozak translation initiation sequence, and “T2A (dme)” are sequences that encode self-cleavage peptide sequences, and “rBG pA” is the rabbit beta-globin polyadenylation signal sequence, “Ampicillin” is an ampicillin resistance gene which allows for selection of the oocytes that have been transposed, and “pUC ori” is the replication origin sequence of the pUC plasmid, which facilitates plasmid replication in the Escherichia coli bacteria.

DNA located between the 5′ ITR and the 3′ ITR sequences may be transposed by a transposase into sites with specific sequences, for example by pBase transposase at sites with TTAA sequence.

DmP2C peptide: refers to the peptide sequence of yolk protein 1 that is recognized by the receptor in oocytes. HyPBase: refers to the improved version of the piggybac transposase.

In those embodiments that include the incorporation of the gene of a recombinant protein of interest in the genetic vector, the present invention allows said recombinant protein gene to be inserted into the genome of oocytes of female insects, enabling the generation of eggs that comprise the gene for said recombinant protein in their genome, and therefore the development of larvae and insects that can express and produce said recombinant protein.

The present invention may be applied with any type of insect.

For example, the present invention may be applied with various species of the mosquito, tick, Drosophila and black soldier fly, among other insects.

As used herein, in an embodiment, the term “female insect” refers to females of any type of insect, such as black soldier fly (Hermetia illucens), Zophobas morio, Tenebrio molitor, fruit fly (Drosophila spp.), green fly (Lucilia sericata), housefly (Musca domestica), bee (Apis spp.), ant (Formicidae spp.), moth (Lepidoptera spp.), bumblebee (Bombus spp.), monarch butterfly (Danaus plexippus), potato beetle (Leptinotarsa decemlineata), rhinoceros beetle (Dynastinae spp.), wax moth (Galleria mellonella), cricket (Gryllidae spp.), dragonfly (Odonata spp.), cockroach (Blattodea spp.), ladybug (Coccinellidae spp.), Japanese rhinoceros beetle (Popillia japonica), mosquito (Culicidae spp.) (Aedes spp.), bed bug (Heteroptera spp.), beetle (Coleoptera spp.), flea (Siphonaptera spp.), termite (Isoptera spp.), mole cricket (Gryllotalpa spp.), wasp (Vespidae spp.), thrips (Thysanoptera spp.), dung beetle (Scarabaeidae spp.), codling moth (Cydia pomonella), scorpion (Scorpions spp.), cicada (Cicadidae spp.), kissing bug (Triatominae spp.), louse (Phthiraptera spp.), tsetse fly (Glossina spp.), American mole cricket (Gryllotalpa hexadactyla), flour moth (Plodia interpunctella), aphid (Aphididae spp.), water bug (Naucoridae spp.), among others.

### Experiments

The following experiments were conducted to show the possibility and the potential efficacy of injecting black flies with a composition that should provide evidence of viability of generating recombinant oocytes in insects and/or arachnids.

### Methodology and Results

### Needles

The following needle types were procured.

### Tuberculin or Insulin:

Brand/material: DB Ultra-Fine 0.5 m|1 U insulin=0.01 mL
Tip length: 0.5 cm (photo with ruler missing)

### Capillaries:

Type/material: No filament/Borosilicate
Tip length: Depends on the puller configuration

### Hamilton:

Type/material: SST/Glass silicate

### Dissection

To become familiar with the anatomy of the fly, one should understand its internal position first. Therefore, sagittal sections were made, as they allow for the individualization of medial elements and their relationship with the anteroposterior and superior-inferior axes. Additionally, axial sections were performed on different individuals, complementing the approach by allowing the relationships of structures on the transverse axis, whether medial or lateral.

### Immobilization:

Fly immobilization occurred using one or more of the following protocols/experiments.
The flies were immobilized with 96% alcohol
They were placed in the refrigerator at 4° C. for 15 minutes.
They were placed in the freezer at −20° C. for 2-3 minutes.
Observation of the anterior and posterior parts was conducted.
Identification of anatomical differences between males and females was also carried out, generating specific distinctions regarding the tail organ, allowing the selection of females.

The following observations were made using the above protocols.

### Observations:

Using the refrigerator at 4° C. does not put the flies completely to sleep, and they wake up very quickly.
Regarding the freezer, 5 minutes was eventually used, but some flies still remained active.
To increase the time the flies remain still, a plate is placed.
It was discovered that the alcohol destroys part of the tissues, making the flies and their tissue difficult to manipulate during dissection.

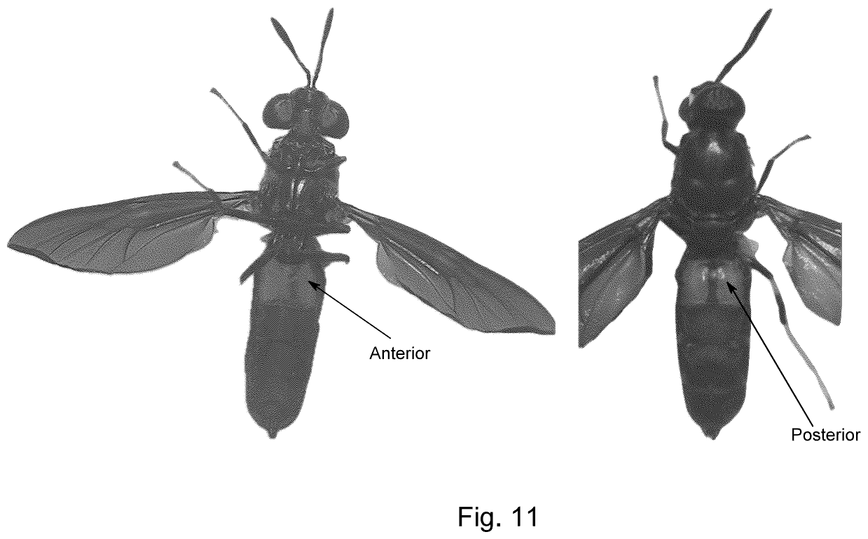

The dissection process began with becoming familiar with the external anatomy of the black soldier flies, as depicted in FIG. 11. This initial step was used to understand the position and orientation of the flies' body parts, which provided guidance during the internal dissection. After gaining a thorough understanding of the external features, the flies' internal anatomy was analyzed to identify differences between male and female flies, focusing on organs that could be affected by various experimental conditions. During the internal dissection, significant challenges were encountered due to the use of alcohol for immobilization. The alcohol not only destroyed external tissues but also affected internal structures, complicating the dissection process and making it difficult to manipulate the tissues accurately.

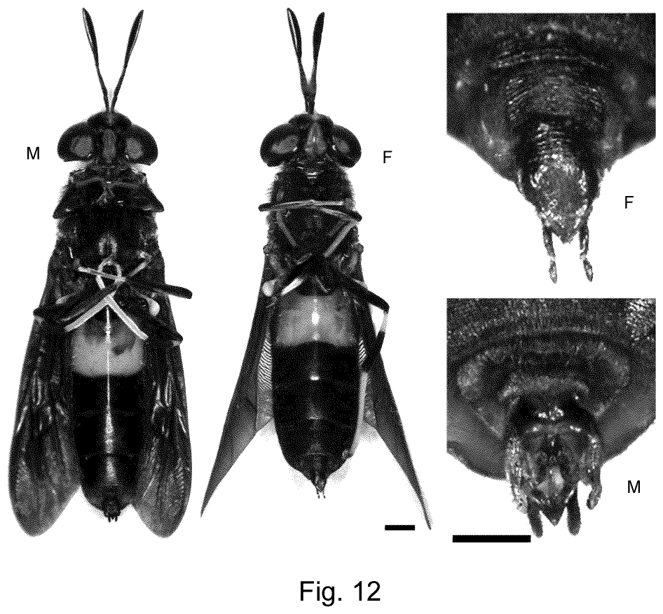

The black soldier fly (Hermetia illucens) exhibits distinct anatomical differences between males and females, particularly in their genitalia. FIG. 12 shows an adult female (F) and male (M) black soldier fly, with magnified views of their genitalia. The female genitalia are adapted for oviposition, making them less prominent and located towards the posterior end of the abdomen. In contrast, the male genitalia are more pronounced and used for mating, with a distinct structure visible when magnified.

### Injection

### Breeding Conditions:

For the breeding of the black soldier flies, the following conditions were maintained in the reproduction room:
Temperature: 28° C.
Humidity: 40-50% RH
Photoperiod: 12/12 (12 hours light, 12 hours dark)
CO₂ concentration: 1000 ppm

### Pupa Selection:

The pupae were selected and kept over a 48-72 hour period. The emergence of adult flies was expected after 96 hours, ensuring that the flies were approximately 24 hours old at the start of the experiments.

### Injection Setup

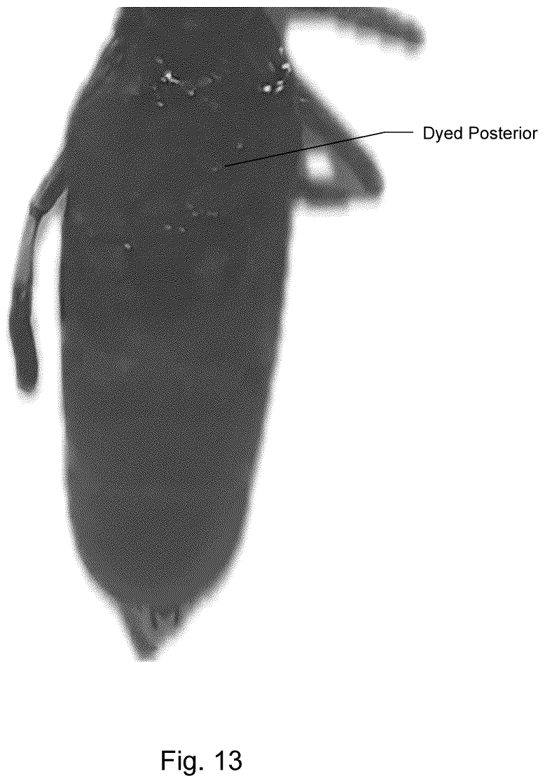

The best method for injection was evaluated, considering the duration the fly remained immobilized as well as potential injection sites. A round of at least 9 flies was injected, with 5 and 4 flies injected in the same parts (at the anterior and posterior windows, respectively). The injection was considered to be successful when the abdomen (see FIG. 13) or back appeared completely fuchsia (from the dye) in color.

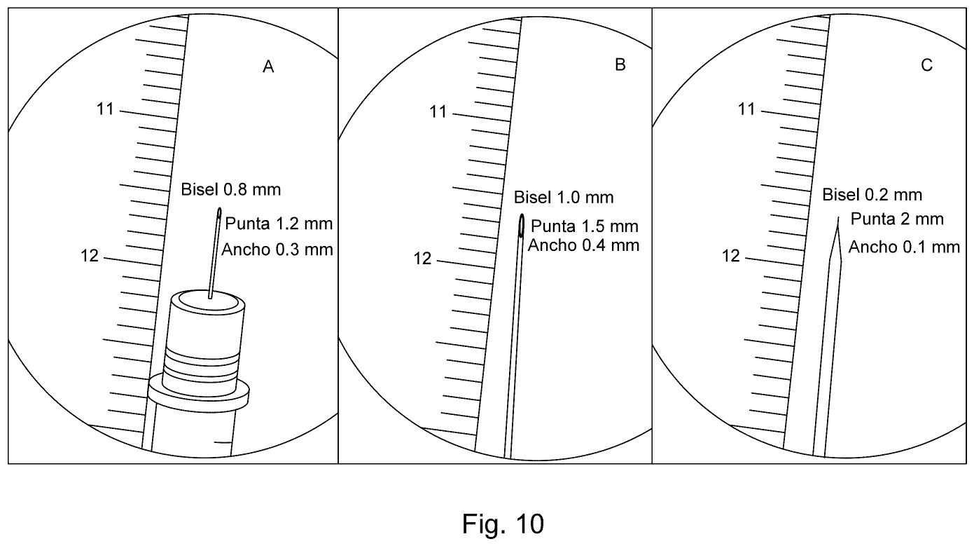

During the experiments, different types of needles were tested for injecting adult flies (see FIG. 10). Initially, injections were performed using the Hamilton needle, but this was found to be not completely satisfactory and was quickly discarded due to its slightly longer bevel, which prevented the liquid from fully entering the insect, resulting in ineffective injections. Capillaries were also attempted, but due to the difficulty in fully immobilizing the insects, the fine tips of the capillaries easily broke, making this option also unviable. Accordingly, insulin/tuberculin needles were chosen, as they proved to be more effective and manageable for the injection procedure.

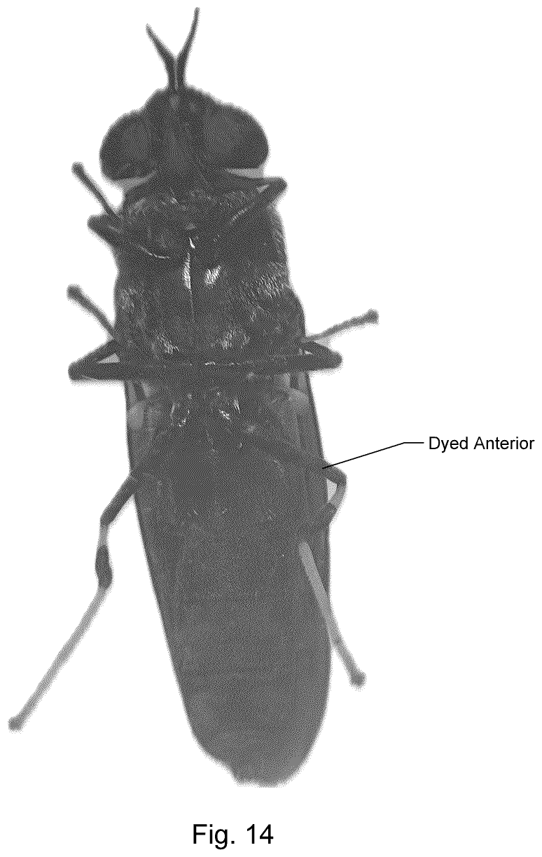

In the first trial, a fly was placed in the freezer for 5 minutes. This initial fly did not open its wings, allowing the injection to be performed through one of the anterior windows with a small volume. This fly, referred to as “Individual 1,” survived and regained mobility within a few minutes. Individual 1 was then placed in a flask with a cotton swab soaked in sugar water at 5% p/v to monitor its survival over the next few days. Notably, the dye used in the first injection trial began to spread to various parts of the fly's body, staining not only the abdomen but also the leg joints as well as the nose (see FIG. 14).

### Experimental Groups:

Eight flies were injected using an insulin needle and placed in the refrigerator for 5 to 15 minutes. However, these flies did not become fully immobilized, even after 15 minutes. It was hypothesized that this might be due to the time of day, as around 5 PM, after spending the entire day in the laboratory, the flies became very active.

Two batches of 8 to 10 female flies were created for each experiment, with one set of flies being injected with an insulin/tubulin needle and the other set serving as the non-injected control group. Each batch was carefully monitored to evaluate the survival rate of the flies on a daily basis.

Starting from the day of injection, the survival of both the injected and control flies was recorded daily. On the third day post-injection, two male flies were added for every female fly in each batch to assess mating behavior and oviposition. This was done to observe any potential effects of the injection on reproductive behavior and success. The males were introduced to both the injected and control groups to ensure consistency across experimental conditions.

The flies were housed in separate containers with a constant supply of sugar water to maintain their health. Observations were made daily to document the number of surviving flies, any mortalities, and any visible changes in behavior or physiology. The data collected included the number of live and dead female flies, as well as the number of males introduced and their survival rate.

### Results

The following two tables 1 and 2 provide a summary of the results that were obtained by employing one of two techniques: the ByRemote technique and the Microinjection technique. The ByRemote technique uses advanced remote tools to deliver genetic material into the oocytes of the ovaries in adult female flies, whereas the microinjection technique involves manually injecting genetic material into the oocytes. As can be seen in tables 1 and 2 below, the ByRemote technique is more efficient, provides generally better results, and provides higher survival rates.

TABLE 1

| | Transformation Rate | |
|---|---|---|
| Technique | (Transformed Individuals/ | Survival to |
| Comparison | Injected Individuals) | Adulthood |
| ByRemote | 33% | 75% |
| Microinjection | 2% | 35% |

As shown in table 1, the first technique, ByRemote, shows a transformation rate of 33% and a survival rate of 75%. In contrast, the microinjection technique has a significantly lower transformation rate of 2% and a survival rate of 35%. This comparison highlights the higher efficiency and survival associated with the ByRemote technique.

TABLE 2
Comparison of Various Parameters associated with the performed
techniques to generate large scale production

| Technique | ByRemote | Microinjection |
|---|---|---|
| Individuals Intervened | 300 | 300 |
| Survivors | 270 | 60 |
| Positive Transformations | 100 | 5 |
| Individuals Obtained | 12,500 | 625 |
| for G1 (approx) | | |
| Eggs Obtained in G2 | 1,562,500 | 78,125 |
| Eggs Obtained in G3 | 195,312,500 | 9,765,625 |
| Eggs Obtained in G4 | 24,414,062,500 | 1,220,703,125 |
| Number of Grams of Eggs | 976,562.5 | 48,828.125 |
| Obtained in G4 | | |

Table 2 provides a comparison of the insects using the two enumerated techniques: ByRemote and Microinjection. Both techniques started with 300 individuals. ByRemote resulted in 270 survivors, with 100 positive transformations. This led to approximately 12,500 individuals for generation 1 (G1), which then produced 1,562,500 eggs in generation 2 (G2). In generation 3 (G3), 195,312,500 eggs were obtained, and by generation 4 (G4), 24,414,062,500 eggs were produced, amounting to a mass of 976,562.5 grams of eggs.

In contrast, the microinjection technique resulted in 60 survivors, with 5 positive transformations. This produced approximately 625 individuals for G1, which led to 78,125 eggs in G2. In G3, 9,765,625 eggs were obtained, and by G4, 1,220,703,125 eggs were produced, amounting to 48,828.125 grams of eggs.

This shows that the ByRemote technique demonstrates significantly higher efficiency, resulting in a far greater number of eggs and grams of eggs obtained by generation 4 compared to the microinjection technique. This shows that employing the ByRemote technique may be unexpectedly and advantageously superior in the mass production of eggs and/or gene products from the transformed oocytes.

### Mortality Count

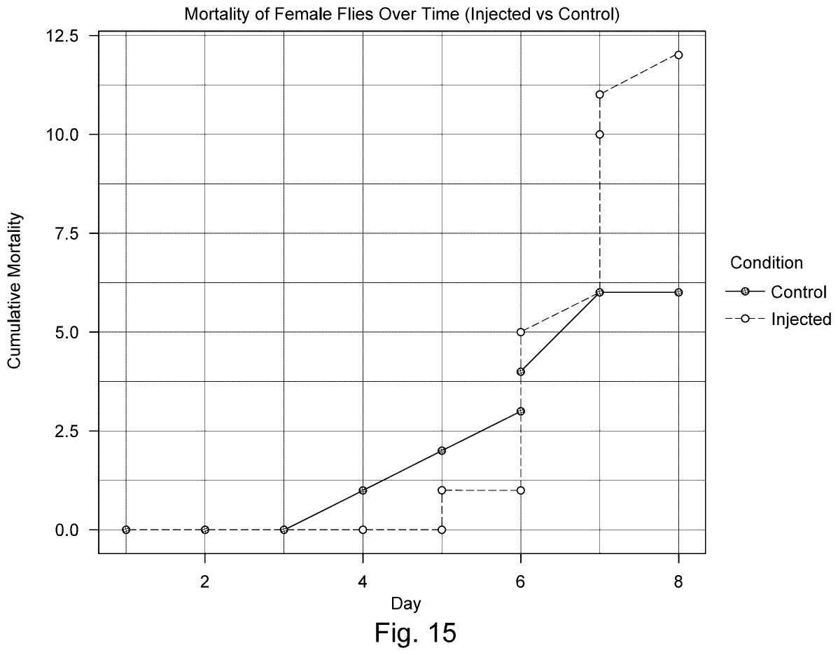

On the first day of observation, both the injected and control groups had a survival rate of 100%. Over the course of the study, the daily survival and mortality rates were monitored, with the survival rates being summarized in FIG. 15. The Kaplan-Meier survival curves show that the survival probability for both groups was nearly identical, indicating no significant difference in survival between the injected and control flies. A calculated p-value of 1 further confirmed that the injection did not significantly affect the survival rate of the flies. The number at risk each day supports this finding, as the counts for both groups decreased at a similar rate.

To assess reproductive behavior and success, we introduced two male flies for each female fly in both groups on the third day post-injection. This step aimed to evaluate mating and oviposition. However, no notable differences in mating behavior or oviposition was noted between the injected and control groups, suggesting that the injection procedure did not adversely affect reproductive success.

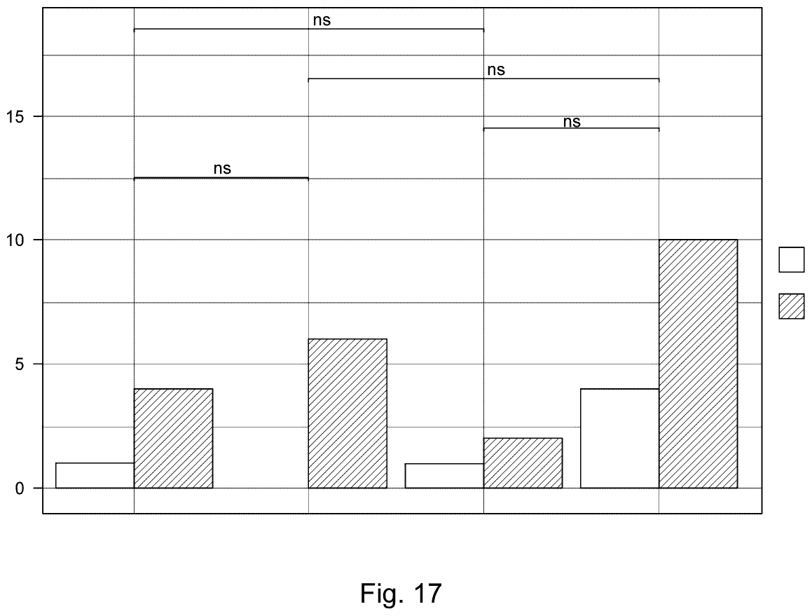

Additionally, FIG. 17 shows the death rates of male and female flies in both the injected and control conditions. It is evident that more males died than females in both conditions. Without being bound by theory, it is believed that this higher mortality rate in males could be attributed to the fact that the male flies had emerged earlier than the female flies. As a result, they began to die naturally due to old age, which is reflected in the increased male mortality over the days.

### Observation of Dye Distribution in Flies

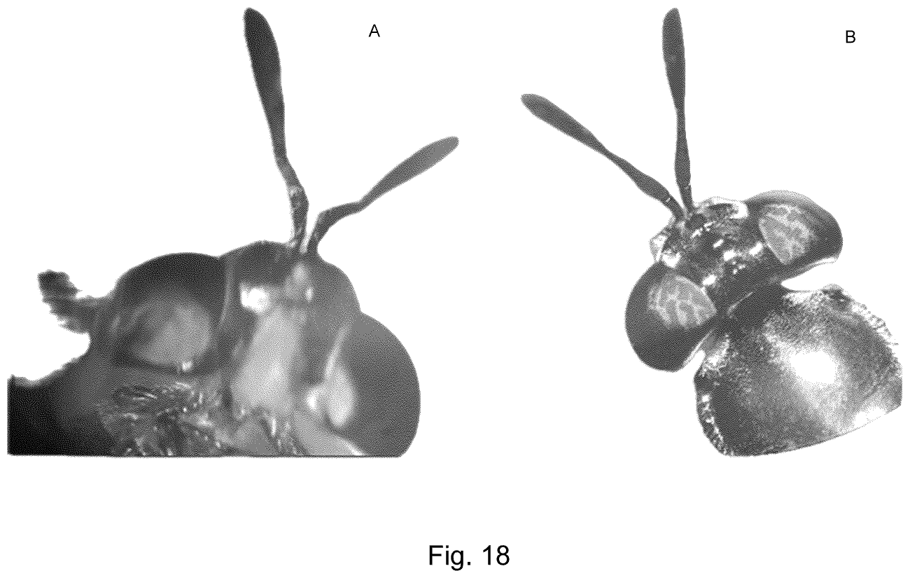

From observations, it was noted that the spread of dye from the injection site to various parts of the flies' bodies occurred over time. Specifically, after two days post-injection, it was observed that the dye had traveled to the antennas of the flies. This indicates that the dye circulates through the hemolymph and reaches distant parts of the body, suggesting a systemic distribution (FIG. 18).

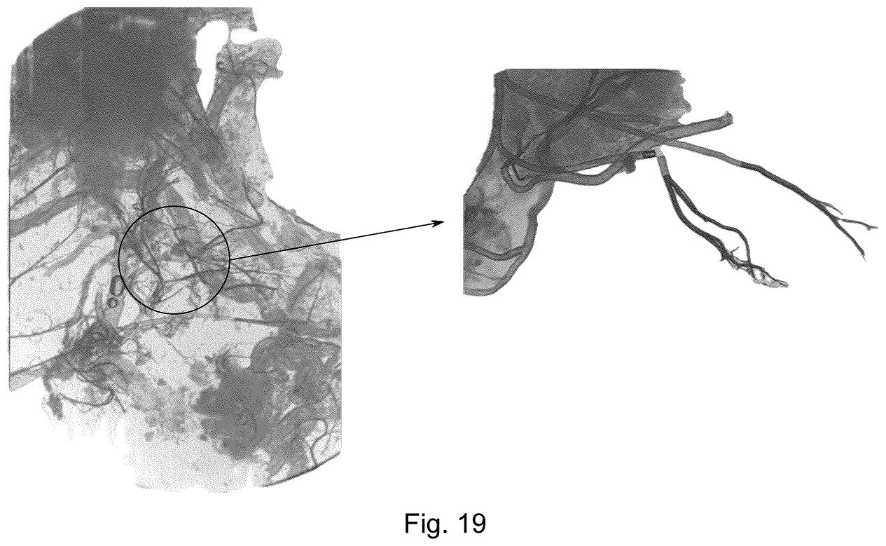

Additionally, the internal distribution of the dye was examined within the flies. Hemolymph, which is an insect's equivalent of blood, was found to be stained several days post-injection, as shown in FIG. 19. This further supports the observation that the injected dye travels through the circulatory system of the fly, reaching various internal organs and tissues.

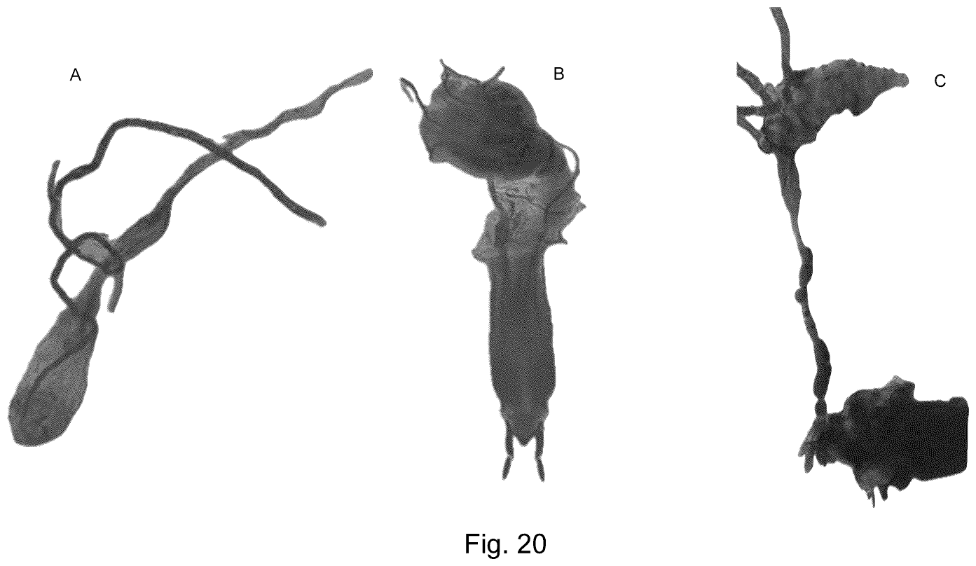

Further detailed dissections were conducted to observe the specific areas affected by the dye at different time points post-injection. FIG. 20A shows internal structures stained with blue dye one hour post injection. FIG. 20B illustrates the bladder and genitalia stained with fuchsia dye from the previous day. Finally, FIG. 20C depicts the digestive system and genitalia of a fly injected three days prior, showing extensive dye distribution.

These observations confirm that the injected dye not only permeates external appendages like antennas but also thoroughly infiltrates internal organs and systems. The progressive staining of different internal structures over time demonstrates the efficiency of dye distribution throughout the fly's body, likely facilitated by the hemolymph circulatory system. This is of utmost importance, as it was confirmed that the transformation vectors are able to enter the hemolymph and thus enter into interaction with the eggs. In these trials, only batch 3 continued with oviposition, so in the future it is hoped that the eggs can be properly analyzed even though they have very low viability.

In an embodiment, the present invention relates to a recombinant oocyte of an insect or arachnid comprising an oocyte and exogenous DNA and/or RNA, said exogenous DNA and/or RNA inserted into the oocyte by use of a peptide, said peptide comprising a P2C peptide that is modified by one or more amino acid insertions, deletions and/or alterations to generate a modified P2C peptide in order to optimize its binding with a vitellogenin receptor of a female insect or arachnid.

By optimized its binding, it is meant that the modified P2C peptide has at least 110% better binding capacity than the unmodified P2C peptide. In alternate embodiments, the binding may be more than 120% better, or more than 130%, or more than 150%, or more than 200%, or more than 250%, or more than 300% better.

In a variation, the modified PC2 peptide comprises a fusion peptide comprising a pBas-PC2 hybrid. In a variation, the modified PC2 peptide is modified by a bound transposase.

In a variation, the exogenous DNA and/or RNA is part of a genetic vector. In a variation, the genetic vector is a vector that further comprises at least one inverted terminal repeat sequence.

In a variation, the insect or arachnid is selected from the group consisting of a mosquito, a tick, a fly, a beetle, a cicada, a termite, a cricket, an aphid, a moth, a dragonfly, a water bug, a butterfly, a bee, a wasp, a cockroach, a ladybug, a bed bug, a flea, and a scorpion.

In a variation, the exogenous DNA and/or RNA comprising a gene of interest that upon expression generates a protein of interest, the gene of interest being located on the genetic vector between several inverted terminal repeat sequences.

In a variation, the bound transposase acts as a gene editing system. In a variation, the genetic vector comprises a gene of interest, more than one inverted terminal repeat sequences, an antibiotic resistant selection gene, a promoter, and a polyadenylation sequence.

In an embodiment, the present invention relates to a method of generating a recombinant oocyte containing exogenous DNA and/or RNA in an insect or arachnid, the method comprising:
a) procuring a P2C peptide;
b) modifying the P2C peptide to generate a modified P2C peptide to optimize its binding with a vitellogenin receptor of a female insect or arachnid;
c) procuring the exogenous DNA and/or RNA and inserting it into a genetic vector; and
as a last step
d) administering the modified P2C peptide and the genetic vector to the female insect or arachnid.

In a variation of the method, the modified PC2 peptide comprises a fusion peptide comprising a pBas-PC2 hybrid. In a variation, the modified PC2 peptide is modified by a bound transposase. In a variation, the genetic vector comprises at least one inverted terminal repeat sequence. In a variation, the exogenous DNA and/or RNA comprises a gene of interest and the genetic vector further comprises the gene of interest, more than one inverted terminal repeat sequences, an antibiotic resistant selection gene, a promoter, and a polyadenylation sequence.

In a variation, the administering step is via injection into the female insect or arachnid. In a variation, the insect or arachnid is selected from the group consisting of a mosquito, a tick, a fly, a beetle, a cicada, a termite, a cricket, an aphid, a moth, a dragonfly, a water bug, a butterfly, a bee, a wasp, a cockroach, a ladybug, a bed bug, a flea, and a scorpion.

In a variation, the genetic vector comprises one or more inverted terminal repeat sequences, a gene that encodes some recombinant protein of interest located between the ITR sequences, a promoter sequence a translation initiation sequence, sequences that encode self-cleavage peptide sequences, a polyadenylation signal sequence, an antibiotic resistance marker gene, and a replication origin sequence.

In a variation, the insect is a black fly. In a variation, the genetic vector is pET28a P2C-HypBase or pET28a P2C-cas9-HypBase.

In a variation, the modified PC2 peptide comprises a fusion peptide comprising a pBas-PC2 hybrid, which further comprises a bound transposase, and wherein the genetic vector comprises one or more inverted terminal repeat sequences, a gene that encodes some recombinant protein of interest located between the ITR sequences, a promoter sequence a translation initiation sequence, sequences that encode self-cleavage peptide sequences, a polyadenylation signal sequence, an antibiotic resistance marker gene, and a replication origin sequence.

As used herein, the term “black soldier fly” refers to insects of the genus Hermetia, including Hermetia illucens.

As used herein, the term “DNA” refers to deoxyribonucleic acid.

As used herein, the term “RNA” refers to ribonucleic acid.

As used herein, the term “gene editing” refers to modifications in nucleic acid sequences that comprise deletions, insertions, mutations and single point modifications among others.

As used herein, the term “transformation” may refer to the incorporation of genetic material, DNA and/or RNA into a living cell, a collection of living cells, such as a tissue or an organ, or a living multicellular organism. The genetic material, DNA and/or RNA, may be incorporated, among other places, within the genome of the cell, the group of living cells or organisms.

As used herein, the term “genetic vector” refers to any type of medium for containing and carrying DNA sequences, such as plasmids, cosmids, artificial chromosomes, among others.

As used herein, the term “CRISPR-Cas9” refers to the set of gene editing methods based on the bacterial defense system related to “clustered regularly interspaced short palindromic repeat” (or CRISPR) sequences of DNA and non-specific CRISPR-associated (Cas) endonuclease proteins, which include several types of Cas proteins and an RNA sequence called guide RNA, which in turn includes a sequence complementary to the target DNA, called “crispr RNA” and an RNA sequence called “trans activating crispr RNA” (or tracr RNA) that serves as a binding site for the Cas endonuclease.

As used herein, the terms “Cas9 protein” or “Cas9” or “Cas” refer to any or all nucleases associated with the CRISPR system, regardless of its/their origin.

As used herein, the term “transposase” refers to an enzyme with transposase activity, that is, it catalyzes the movement of a DNA fragment from one location to another between or within one or more of a genome, a genetic vector, a chromosome, among other genetic elements.

As used herein, the term “genetic element” refers to a nucleic acid molecule with a particular sequence of nucleotides.

In the present invention, when the generation or construction of certain elements is mentioned, such as genetic vectors, fusion proteins among others, or the fusion of certain proteins or peptides to generate fusion proteins, it is understood that this is done by means of methods, techniques and procedures known in the state of the art, which a person of ordinary skill in the art could know and implement. For this reason, these methods, techniques and procedures are not detailed herein.

It should be understood that definitions of terms and expressions used in more than one aspect of the invention apply equally to any aspect of the invention described herein in which those terms and expressions appear. This applies equally regardless of where, that is, in what section, within this application, such terms are defined or discussed.

Throughout this document, where compositions are described as having, including, or comprising specific components, they are considered to have the same meaning. Likewise, where processes are described as having, including, or comprising specific process steps, they are considered to have the same meaning. Where compositions of the present teachings are considered to consist essentially of the mentioned components, and the processes of the present teachings also consist essentially of the mentioned process steps, they are considered to have the meaning which are known to those in the patent field.

Where an element or component is described as being selected from a list of mentioned elements or components or otherwise known to express Markush language, it should be understood that the element or component may be any of the mentioned elements or components, or that the element or component may be selected from a group of two or more of the aforementioned elements or components. Furthermore, it is contemplated by the inventors of the present invention and it should be understood that the elements and/or characteristics of a composition, apparatus or method described herein may be combined in a variety of ways even if they are not expressly mentioned together, without departing from the spirit and scope of the present invention, whether explicitly or implicitly herein. For example, when reference is made to a particular structure, that structure may be used in various embodiments of the apparatus of the present teachings and/or in the methods of the present teachings and combined with other structure or apparatuses, unless otherwise understood by the context.

The use of the singular form, for example, “a”, “an”, and “the”, may include the plural (and vice versa) unless specifically indicated otherwise.

The use of the terms “includes”, “which includes”, “including”, “has/have”, “which has/have”, “having”, “contains”, “which contains”, “containing”, “which comprises” or “comprising”, including the grammatical equivalents thereof, should be understood generally as open and non-limiting, for example, without excluding additional unmentioned elements or steps, unless otherwise specifically indicated or understood by the context.

It should be understood that the expressions “one of” or “at least one of”, and the grammatical equivalents thereof, individually include each of the objects, elements or substances mentioned after the expression, and the different combinations of two or more of the objects, elements or substances mentioned, unless otherwise understood by context and use.

Although the present application mentions separate embodiments, it should be understood that any embodiment, and the characteristic features thereof, can be freely combined with any other embodiment and the characteristic features thereof, even in the absence of an explicit statement to this effect.

It should be understood that the order of steps or the order of performing certain actions is irrelevant as long as the present teachings remain operative unless the order is specifically and explicitly stated to occur in a given order. Additionally, two or more steps or actions can be carried out simultaneously unless it is specifically and explicitly stated that they do not occur simultaneously.

The use of any and all examples or exemplary language herein, such as “such as” or “including,” is intended only to better illustrate the teachings herein and is not intended to limit the scope of the invention, unless so declared. No expression in the specification should be construed as indicating that any unclaimed element is considered essential to the practice of the present teachings.

Even though the elements, compounds, formulations, compositions, methods and uses of this invention have been described in terms of embodiments or examples, it will be apparent to those skilled in the art that variations may be applied to the elements, compounds, formulations, compositions, methods and uses, as well as in the steps or sequences of steps of the methods described herein without departing from the concept, spirit and scope of the invention. More specifically, it will be apparent that certain agents that are both chemically and physiologically related may substitute for the agents described herein while achieving the same or similar results. All substitutes and similar modifications apparent to those skilled in the art are considered to be within the spirit, scope and concept of the invention as defined.

Any reference referred to herein is incorporated by reference in its/their entirety for all purposes.

We claim:

1. A recombinant oocyte of an insect or arachnid comprising an oocyte and exogenous DNA and/or RNA, said exogenous DNA and/or RNA inserted into the oocyte by use of a peptide, said peptide comprising a P2C peptide that is modified by one or more amino acid insertions, deletions and/or alterations to generate a modified P2C peptide in order to optimize its binding with a vitellogenin receptor of a female insect or arachnid.

2. The recombinant oocyte of claim 1, wherein the modified PC2 peptide comprises a fusion peptide comprising a pBas-PC2 hybrid.

3. The recombinant oocyte of claim 1, wherein the modified PC2 peptide is modified by a bound transposase.

4. The recombinant oocyte of claim 1, wherein the exogenous DNA and/or RNA is part of a genetic vector.

5. The recombinant oocyte of claim 4, wherein the genetic vector is a vector that further comprises at least one inverted terminal repeat sequence.

6. The recombinant oocyte of claim 1, wherein the insect or arachnid is selected from the group consisting of a mosquito, a tick, a fly, a beetle, a cicada, a termite, a cricket, an aphid, a moth, a dragonfly, a water bug, a butterfly, a bee, a wasp, a cockroach, a ladybug, a bed bug, a flea, and a scorpion.

7. The recombinant oocyte of claim 5, the exogenous DNA and/or RNA comprising a gene of interest that upon expression generates a protein of interest, the gene of interest being located on the genetic vector between several inverted terminal repeat sequences.

8. The recombinant oocyte of claim 3, wherein the bound transposase acts as a gene editing system.

9. The recombinant oocyte of claim 5, wherein the genetic vector comprises a gene of interest, more than one inverted terminal repeat sequences, an antibiotic resistant selection gene, a promoter, and a polyadenylation sequence.

10. A method of generating a recombinant oocyte containing exogenous DNA and/or RNA in an insect or arachnid, the method comprising:
a) procuring a P2C peptide;
b) modifying the P2C peptide to generate a modified P2C peptide to optimize its binding with a vitellogenin receptor of a female insect or arachnid;
c) procuring the exogenous DNA and/or RNA and inserting it into a genetic vector; and as a last step
d) administering the modified P2C peptide and the genetic vector to the female insect or arachnid.

11. The method of claim 10, wherein the modified PC2 peptide comprises a fusion peptide comprising a pBas-PC2 hybrid.

12. The method of claim 10, wherein the modified PC2 peptide is modified by a bound transposase.

13. The method of claim 10, wherein the genetic vector comprises at least one inverted terminal repeat sequence.

14. The method of claim 13, wherein the exogenous DNA and/or RNA comprises a gene of interest and the genetic vector further comprises the gene of interest, more than one inverted terminal repeat sequences, an antibiotic resistant selection gene, a promoter, and a polyadenylation sequence.

15. The method of claim 10, wherein the administering step is via injection into the female insect or arachnid.

16. The method of claim 10, wherein the insect or arachnid is selected from the group consisting of a mosquito, a tick, a fly, a beetle, a cicada, a termite, a cricket, an aphid, a moth, a dragonfly, a water bug, a butterfly, a bee, a wasp, a cockroach, a ladybug, a bed bug, a flea, and a scorpion.

17. The method of claim 12, wherein the genetic vector comprises one or more inverted terminal repeat sequences, a gene that encodes some recombinant protein of interest located between the ITR sequences, a promoter sequence a translation initiation sequence, sequences that encode self-cleavage peptide sequences, a polyadenylation signal sequence, an antibiotic resistance marker gene, and a replication origin sequence.

18. The method of claim 16, wherein the insect is a black fly.

19. The method of claim 10, wherein the genetic vector is pET28a P2C-HypBase or pET28a P2C-cas9-HypBase.

20. The method of claim 10, wherein the modified PC2 peptide comprises a fusion peptide comprising a pBas-PC2 hybrid, which further comprises a bound transposase, and wherein the genetic vector comprises one or more inverted terminal repeat sequences, a gene that encodes some recombinant protein of interest located between the ITR sequences, a promoter sequence a translation initiation sequence, sequences that encode self-cleavage peptide sequences, a polyadenylation signal sequence, an antibiotic resistance marker gene, and a replication origin sequence.
